(19) United States
(12) Patent Application Publication
(10) Pub. No.: US 2007/0259976 A1
(43) Pub. Date: Nov. 8, 2007

(54) Catalyst for the Conversion of Carbon Monoxide

(76) Inventors: Hiroshi Takeda, Nei-gun (JP); Troy Walsh, Louisville, KY (US); Jon P. Wagner, Louisville, KY (US)

Correspondence Address: SUD-CHEMIE INC., 1600 WEST HILL STREET, LOUISVILLE, KY, 40210, US

(21) Appl. No.: 11/381,938

(22) Filed: May 5, 2006

(51) Int. Cl. C07C 27/06

(52) U.S. Cl. 518/716

(57) ABSTRACT

Use of a catalyst composition comprising a metal selected from the group consisting of ruthenium, rhodium, nickel and combinations thereof, on a support selected from the group consisting of a beta-zeolite, mordenite and faujasites, is taught for carbon oxide methanation reactions for fuel cells. Specifically, when a mixture of gases containing hydrogen, carbon dioxide, carbon monoxide, and water is passed over the catalyst in a reaction zone having a temperature below the temperature at which the shift reaction occurs and above the temperature at which the selective methanation of carbon monoxide occurs, the catalyst efficiently facilitates the selective hydrogenation of carbon monoxide using H₂ that is present in the reformate and reduces the concentration of the CO to levels equal to or less than about 50 ppm and demonstrates a carbon monoxide (CO) methanation selectivity of greater than about 50%.

### CROSS-REFERENCE TO RELATED APPLICATIONS

The present application is related to U.S. application Ser. No. 10/740,144 filed on Dec. 18, 2003 and incorporated herein in its entirety by reference.

### BACKGROUND OF THE INVENTION

The present invention is the use of a specific catalyst composition for carbon oxide methanation reactions for fuel cells. Specifically, when a mixture of gases containing hydrogen, carbon dioxide, carbon monoxide, and water is passed over the catalyst in a reaction zone having a temperature below the temperature at which the shift reaction occurs and above the temperature at which the selective methanation of carbon monoxide occurs, the catalyst efficiently facilitates the selective hydrogenation of carbon monoxide using H₂ that is present in the reformate and reduces the concentration of the CO to levels equal to or less than about 50 ppm and demonstrates a carbon monoxide (CO) methanation selectivity of greater than about 50%. This is a significant improvement over selective methanation catalysts of the prior art.

In a fuel cell, such as a Polymer Electrolyte Membrane Fuel Cell (PEMFC) stack, chemical energy of a fuel is converted into electrical energy. Typically, the fuel used is a hydrogen rich gas supplied to the fuel cell by a fuel processor. However, the gas from the fuel processor may further comprise unconverted hydrocarbon, water, carbon dioxide and carbon monoxide. The carbon monoxide, in particular, is detrimental to the PEMFC stack because the carbon monoxide can poison the noble metal electrodes utilized by the fuel cells, thereby reducing the electrical output.

Preferably, the CO concentration for a fuel cell feed should be at a level below about 100 ppm, and more preferably to a level of less than about 50 ppm. However, as received from the fuel processor, the CO concentrations may be in excess of about 1 wt %, thus requiring further reduction of CO concentration. Some typical methods for reducing the CO concentration include selective catalytic oxidation of CO, pressure swing adsorption, hydrogen separation by membrane, and selective methanation of CO.

Selective catalytic oxidation of CO (Eq. 1) is a well-known process for reducing the CO concentration for fuel cells. But, oxidation of hydrogen (Eq. 2) is a competitive reaction.
½O₂+CO→CO₂ Eq. 1
½O₂+H₂→H₂O Eq. 2
Thus, in order to maximize the concentration of hydrogen gas and minimize the concentration of carbon monoxide, it is necessary to have reaction conditions wherein Eq. 1 is favored over Eq. 2. One option for achieving this is to have a highly specific catalyst for the oxidation of carbon monoxide and to limit the oxygen concentration so that the oxygen is consumed primarily for the production of carbon dioxide. Theoretically, this is achievable, but in practice there are wide swings in the CO concentrations produced by the fuel processor and it can be difficult to adjust the oxygen input to track the CO concentration. Because the CO is more detrimental to the fuel cell than water, it is typical for excess oxygen to be fed into the reactor thereby essentially ensuring that the CO will be converted to CO₂. The disadvantage is that significant quantities of H₂ are converted to water by operating in this manner.

Pressure swing adsorption is an industrially proven technology, but it requires relatively high pressure operation. Thus, while this process may be effective for use in larger fuel cells, it is not practical at this time for smaller fuel cells.

Hydrogen separation by membrane is effective for separating hydrogen from carbon monoxide. But the process requires a substantial pressure drop to effect the separation, and the cost and durability of the membranes still must be proven.

Selective methanation (Eq. 3) is a process whereby carbon monoxide is reacted with hydrogen in the presence of a catalyst to produce methane and water and methanation of carbon dioxide is minimized. Commonly used in ammonia plants, total carbon oxide methanation is known to reduce carbon monoxide and carbon dioxide concentrations to levels as low as about 5 ppmv to 10 ppmv, and the industrial catalysts are not selective. However, in most fuel cell applications, the selective methanation reaction is accompanied by a reverse water-gas-shift reaction (Eq. 4), which also is generally facilitated by a catalyst. Thus, while the CO concentration is being reduced through methanation, additional carbon monoxide is formed from the carbon dioxide present to maintain the equilibrium of the water-gas-shift reaction.
CO+3H₂→CH₄+H₂O Eq. 3
CO₂+H₂ [symbol] CO+H₂O Eq. 4
Under the proper reaction conditions and with a non-selective methanation catalyst, the CO₂ may be methanated as shown in Eq. 5.
CO₂+4H₂→CH₄+2H₂O Eq. 5
But, this is generally an undesirable reaction because it further consumes H₂ and the CO₂ methanation is normally accompanied by a temperature rise in the reactor that can lead to “run-away” conditions. Considering that the carbon dioxide concentration is greater than 10 times that of carbon monoxide, achieving selectivity is not thermodynamically favorable. Thus, it would be advantageous to have a catalyst that is highly selective for CO methanation, essentially suppresses CO₂ methanation and does not facilitate the conversion of CO₂ to CO through the water-gas-shift reaction.

In the prior art methanation processes, precious metals supported on non-zeolitic materials, such as Al₂O₃, SiO₂, and TiO₂, have been used as catalysts in the selective methanation of CO (see, for example, U.S. Pat. No. 3,615,164 and U.S. Pat. Pub. No. 2003/0086866). For example, in Patent Number WO 01/64337, ruthenium (Ru) on a carrier base support of Al₂O₃, TiO₂, SiO₂, ZrO₂, or Al₂O₃—TiO₂ with egg-shell structure is taught to reduce the CO to concentrations of about 800 ppm with 70-80% selectivity under an atmosphere of CO at 0.6%, CO₂ at 15%, H₂ at 64.4%, H₂O at 20% and GHSV=10,000 H⁻¹. However, for an efficient PEMFC power system, the CO concentration should be less than about 100 ppm, and preferably equal to or less than about 50 ppm. Since the CO concentration from the selective methanation processes using the prior art catalysts are significantly higher than the desired maximum concentration for a PEMFC stack, these catalysts cannot be practically used in PEMFC power systems.

Thus, it would be advantageous to have a catalyst that is highly selective for CO methanation, essentially suppresses CO₂ methanation and does not facilitate the conversion of CO₂ to CO through the water-gas-shift reaction.

### SUMMARY OF THE INVENTION

The present invention is the use of a catalyst comprising a metal that can form a metal-carbonyl species on a support having a regular lattice structure and a predetermined pore diameter of sufficient dimensions to accommodate the carbonylated metal species for carbon oxide methanation reactions for fuel cells. More specifically, the catalyst comprises a metal selected from the group consisting of ruthenium, rhodium, nickel and combinations thereof, on a support selected from the group consisting of a beta-zeolite, mordenite and faujasite. An inert binder, such as alumina, γ-Al₂O₃, SiO₂, ZrO₂, TiO₂ or pseudo-boehmite, may optionally be added to the catalyst.

When a mixture of gases containing hydrogen, carbon dioxide, carbon monoxide, and water is passed over the catalyst in a reaction zone having a temperature below the temperature at which the shift reaction occurs and above the temperature at which the selective methanation of carbon monoxide occurs, the catalyst efficiently facilitates the selective hydrogenation of carbon monoxide using H₂ that is present in the reformate and reduces the concentration of the CO to levels equal to or less than about 50 ppm and demonstrates a carbon monoxide (CO) methanation selectivity of greater than about 50%. This is a significant improvement over selective methanation catalysts of the prior art.

### DETAILED DESCRIPTION OF THE PREFERRED EMBODIMENT

Carbon oxide methanation reactions in small fuel cells can be facilitated by using a catalyst having a predetermined pore size of sufficient dimensions to allow the pore to accommodate a fully carbonylated metal complex. The methanation reaction is a process for reducing the quantity of carbon monoxide in a mixture of gases containing hydrogen and carbon monoxide. The process of the present invention comprises passing a feedstream containing gases selected from hydrogen, carbon dioxide, carbon monoxide, water and combinations thereof over the catalyst in a reactor reaction zone at a temperature of from about 150° C. to about 300° C. and at a gas flow rate of from about 2,000 vol/vol/hr to about 40,000 vol/vol/hr. More specifically, the catalyst comprises a metal selected from the group consisting of ruthenium, rhodium, nickel and combinations thereof, on a support selected from the group consisting of a beta-zeolite, mordenite and faujasite. Optionally, the catalyst may comprise an inert binder, such as a binder selected from the group consisting of alumina, γ-Al₂O₃, SiO₂, ZrO₂, TiO₂, pseudo-boehmite, and combinations thereof.

As is known in the art, some typical supports for catalysts are crystalline alumino-silicate materials. Among the metals known in the art to form stable metal-carbonyl complexes are ruthenium, rhodium, nickel, iron, cobalt, rhenium, palladium, lead and tin, as an exemplary group. Optionally, an inert binder, such as alumina, γ-Al₂O₃, SiO₂, ZrO₂, TiO₂ or pseudo-boehmite, may optionally be added to the catalyst.

The present invention will be described herein through, without limitation, exemplary embodiments, figures and examples. Any embodiments, figures, examples and related data presented herein are merely to exemplify the principles of the invention, and are not intended to limit the scope of the invention.

The support of the catalyst of the present invention comprises a crystalline alumino-silicate having a predetermined pore size. More specifically, the crystalline alumino-silicate can be a molecular sieve, beta-zeolite, mordenite, faujasite or any other alumino-silicate with a regular lattice structure. Other supports that also have regular lattice structures and essentially consistent pore sizes that may be used in place of the alumino-silicate for the catalyst of the present invention include alumina, titania, ceria, zirconia and combinations thereof. Because it is believed that the methanation reaction occurs within the support pore, the pore must be of sufficient dimensions to accommodate a fully carbonylated metal complex, and thus, the pore size requirement will vary depending on the metal species selected for the catalyst. However, it has generally been observed that if the pore size is smaller than or is significantly larger than the dimensions of the fully carbonylated metal species, the resulting catalyst does not show the desired selectivity for carbon monoxide methanation.

The metal of the catalyst of the present invention must be capable of forming a metal-carbonyl species. As is known in the art, metals may form metal-carbonyl complexes wherein each ligand is a carbonyl unit, such as Fe(CO)₅, or metals may form metal-carbonyl complexes wherein at least one ligand is not a carbonyl, such as CpFe(CO)₃. For the purpose of the development, it is not necessary that the metal be capable of forming a fully-carbonylated complexes, e.g. wherein each ligand is a carbonyl group. Rather, a “fully-carbonylated” complex—for the purpose of calculating the volume needed within the support pore—is defined herein as the metal complex with the maximum number of carbon monoxide ligands that the metal prefers to accommodate in its lowest energy state. The metal is preferably selected from the group consisting of ruthenium, rhodium, platinum, palladium, rhenium, nickel, iron, cobalt, lead, tin, silver, iridium, gold, copper, manganese, zinc, zirconium, molybdenum, other metals that form a metal-carbonyl species and combinations thereof. As delivered to the catalyst, the metal may be a base metal or it may be a metal oxide complex.

The metal may be added to the support by any means known in the art for intercalating the metal into the support pores, such as, without limitation, impregnation, incipient wetness method, immersion and spraying. The embodiments presented herein add the metal through impregnation for exemplary purposes only. Although not a requirement to practice the invention, it is recommended that the metal source be free of typically recognized poisons, such as sulfur, chlorine, sodium, bromine, iodine or combinations thereof. Acceptable catalyst can be prepared using metal sources that include such poisons, but care must be taken to wash the poisons from the catalyst during production of the catalyst.

In an embodiment of the present invention, the support is a crystalline alumino-silicate selected from mordenite, beta-zeolite or faujasite. The support has a pore diameter of greater than about 6.3 Å, and a pore volume in the range of from about 0.3 cm³/g to about 1.0 cm³/g, and preferably in the range of 0.5 cm³/g to about 0.8 cm³/g. Ruthenium is impregnated on the support so as to deliver a concentration of from about 0.5 wt % Ru to about 4.5 wt % Ru, based on the total weight of the catalyst including the ruthenium. Some recommended sources of ruthenium include, without limitation, Ru(NO)(NO₃)ₓ(OH)_{y}, Ru(NO₂)₂(NO₃)₂, Ru(NO₃)₃, RuCl₃, Ru(CH₃COO₃), (NH₄)₂RuCl₆, [Ru(NH₃)₆]Cl₃, Ru(NO)Cl₃, and Ru₃(CO)₁₂. Optionally, the catalyst further comprises the binder γ-Al₂O₃ at a loading of about 20 wt %, including the weight of the binder.

The catalyst may be used in an exemplary process for removing or substantially reducing the quantity of carbon monoxide in a mixture of gases containing hydrogen, carbon dioxide, carbon monoxide, and water. For example, an exemplary feedstream comprises hydrogen at a concentration of from about 30% to about 80%, preferably from about 40% to about 70%, on a dry gas basis; CO₂ at a concentration of from about 0.1% to about 25%, preferably from about 0.25% to about 17%, on a dry gas basis; CO at a concentration of from about 0.1% to about 1.0%, preferably from about 0.25% to about 0.75%, on a dry gas basis; and H₂O at a concentration of from about 0.5% to about 50%, and preferably from about 5.0% to about 35%. The process of the present invention comprises passing a feedstream containing gases selected from hydrogen, carbon dioxide, carbon monoxide, water and combinations thereof over the catalyst in a reactor reaction zone at a temperature of from about 150° C. to about 300° C., and preferably from 175° C. to about 250° C. In this temperature range, the catalyst efficiently facilitates the selective hydrogenation of carbon monoxide using H₂ that is present in the reformate and reduces the concentration of the CO to levels equal to or less than about 50 ppm and demonstrates a carbon monoxide (CO) methanation selectivity of greater than about 50%. The process is preferably carried out at a gas flow rate—as defined as the volumetric flow rate at standard temperature and pressure (0 C, 1 atm) divided by the catalyst volume (Space Velocity)—of from about 2,000 vol/vol/hr to about 40,000 vol/vol/hr, and preferably from about 5,000 vol/vol/hr to about 10,000 vol/vol/hr. The pressure may range from about 1 atm to about 50 bar.

It is understood that variations may be made which would fall within the scope of this development. For example, although the catalysts of the present invention are intended for use as selective methanation catalysts for the conversion of carbon monoxide for fuel cell applications, it is anticipated that these catalysts could be used in other applications requiring highly selective carbon oxide methanation catalysts.

What is claimed is:

1. A process for reducing the quantity of carbon monoxide in a mixture of gases containing hydrogen and carbon monoxide wherein the process comprises passing a feedstream containing gases selected from hydrogen, carbon dioxide, carbon monoxide, water and combinations thereof over a catalyst in a reactor reaction zone, and wherein said catalyst comprises a metal selected from the group consisting of ruthenium, rhodium, nickel and combinations thereof, on a support selected from the group consisting of a beta-zeolite, mordenite and faujasite.

2. The process of claim 1 wherein said feedstream contacts said catalyst at a temperature of from about 150° C. to about 300° C., and at a flow rate of from about 2,000 vol/vol/hr to about 40,000 vol/vol/hr.

3. The process of claim 1 wherein said catalyst further comprises an inert binder selected from the group consisting of alumina, γ-Al₂O₃, SiO₂, ZrO₂, TiO₂ or pseudo-boehmite.

4. The process of claim 1 wherein the metal of the catalyst is ruthenium, and the ruthenium is impregnated on the support so as to deliver a concentration of from about 0.5 wt % Ru to about 4.5 wt % Ru, based on the total weight of the catalyst including the ruthenium.

5. The process of claim 1 wherein said catalyst support has a pore diameter of greater than about 6.3 Å and a pore volume in the range of from about 0.3 cm³/g to about 1.0 cm³/g.

6. The process of claim 1 wherein said catalyst comprises a metal capable of forming a metal-carbonyl species on a support having a pore volume in the range of from about 0.3 cm³/g to about 1.0 cm³/g

7. A process for reducing the quantity of carbon monoxide in a mixture of gases containing hydrogen and carbon monoxide wherein the process comprises passing a feedstream containing gases selected from hydrogen, carbon dioxide, carbon monoxide, water and combinations thereof over a catalyst in a reactor reaction zone, wherein said feedstream contacts said catalyst at a temperature of from about 150° C. to about 300° C., and at a flow rate of from about 2,000 vol/vol/hr to about 40,000 vol/vol/hr, and wherein said catalyst comprises ruthenium impregnated on a support selected from the group consisting of a beta-zeolite, mordenite and faujasite, and the ruthenium is impregnated on the support so as to deliver a concentration of from about 0.5 wt % Ru to about 4.5 wt % Ru, based on the total weight of the catalyst including the ruthenium.

8. The process of claim 7 wherein the catalyst further comprises an inert binder selected from the group consisting of alumina, γ-Al₂O₃, SiO₂, ZrO₂, TiO₂ or pseudo-boehmite.

9. A process for reducing the quantity of carbon monoxide in a mixture of gases containing hydrogen and carbon monoxide wherein the process comprises passing a feedstream containing gases selected from hydrogen, carbon dioxide, carbon monoxide, water and combinations thereof over a catalyst in a reactor reaction zone, and wherein said catalyst is prepared by reacting a metal selected from the group consisting of ruthenium, rhodium, nickel and combinations thereof, with a support having a pore volume in the range of from about 0.3 cm³/g to about 1.0 cm³/g, and then oven-drying the metal-treated support and then calcining the metal-treated support.

10. The process of claim 9 wherein the catalyst support is selected from the group consisting of a crystalline alumino-silicate, a molecular sieve, beta-zeolite, mordenite, faujasite, any other alumino-silicate with a regular lattice structure, alumina, titania, ceria, zirconia and combinations thereof.

11. The process of claim 9 wherein said catalyst further comprises a binder selected from the group consisting of alumina, γ-Al₂O₃, SiO₂, ZrO₂, TiO₂ and pseudo-boehmite, wherein the binder is added by mixing with the support.

12. The process of claim 9 wherein the catalyst metal is ruthenium impregnated on the support so as to deliver a concentration of from about 0.5 wt % Ru to about 4.5 wt % Ru, based on the total weight of the catalyst including the ruthenium.

13. The process of claim 9 wherein the catalyst support has a pore diameter of greater than about 6.3 Å.

14. The process of claim 9 wherein the catalyst further comprises a binder selected from the group consisting of alumina, γ-Al₂O₃, SiO₂, ZrO₂, TiO₂ and pseudo-boehmite, at a loading of about 20 wt %, including the weight of the binder, wherein the binder is added by mixing with the support.
